(19) United States
(12) Patent Application Publication
(10) Pub. No.: US 2023/0256395 A1
(43) Pub. Date: Aug. 17, 2023

(54) DEVICE AND METHOD FOR EXTRACTING BIOMOLECULES

(71) Applicant: LABTURBO BIOTECH CORPORATION, Taipei City (TW)

(72) Inventors: Chi-Sheng TAI, TAIPEI CITY (TW); Shih Jeng DAI, TAIPEI CITY (TW)

(21) Appl. No.: 18/078,094

(22) Filed: Dec. 9, 2022

(30) Foreign Application Priority Data
Feb. 16, 2022 (TW) 111105538

(51) Int. Cl. B01D 63/02

(52) U.S. Cl. CPC B01D 63/021; B01D 2313/04

(57) ABSTRACT

A device for extracting biomolecules includes a membrane tube and a container. The membrane tube has an upper portion and a lower portion, wherein the upper portion has an open end and the lower portion contains permeable membranes and also has an open end. The container has an upper portion and a lower portion, wherein the upper portion has an open end, allowing the membrane tube to be pushed into the container and form a liquid-tight seal between the membrane tube and the container, and the lower portion has a closed end.

### FIELD OF THE INVENTION

The present invention relates to a device and a method for extracting biomolecules.

### BACKGROUND

Membrane tubes are often used to extract specific proteins, nucleic acids, and other biomolecules in biological samples. A membrane tube is of a tubular shape with a permeable membrane contained therein. The upper portion of the membrane tube has an opening, and the lower portion accommodates the permeable membrane and has a lower opening. In the traditional way of using a membrane tube, liquid is added to the membrane tube and then centrifugal force generated by a centrifuge or a pressure difference generated by a vacuum pump is applied to the membrane tube. The method of carrying out the extraction generally includes three basic processes:
1. Accumulation: Add a liquid sample into the membrane tube, and apply appropriate force to push the liquid sample through the lower opening of the membrane tube. During the accumulation process, specific biomolecules in the liquid sample will be accumulated on the membrane.
2. Washing: Add cleaning solution to the membrane tube and apply force to let the remaining impurities in the membrane tube pass through the membrane and be exhausted from the membrane tube.
3. Recovering: Add dissolving solution to the membrane tube to dissolve the target biomolecules on the membrane, and apply force to push them out of the tube and enter another new container to achieve the purpose of recovering the target biomolecules.

The above-mentioned traditional operating method needs to utilize devices such as a centrifuge or vacuum pump to generate force to push the liquid in the membrane tube to flow out through the membrane to the lower opening, to carry out processes such as accumulation, washing, and recovery. The operation of the centrifuge device is relatively complicated and not suitable for processing a large number of specimens. If a vacuum pump is used in a recovery process, a dedicated vacuum pump different from that used in the accumulation and washing processes should be used to ensure that the recovery process is carried out under pollution-free conditions. However, an additional set of vacuum pumps will increase some of the difficulties in the design and implementation of the automation equipment.

### SUMMARY OF INVENTION

The objective of the present invention is to develop a method for simplifying the processing of specimens and an apparatus for applying the method to facilitate automated processing. In the present invention, the squeezing force generated when the membrane tube is pushed into the container makes the liquid in the container enter the membrane tube through the lower opening thereof. In the operation method, an appropriate amount of liquid is added into a container, and then the membrane tube is pushed into the container and a liquid-tight seal is formed between the membrane tube and the container. As the membrane tube continues to be pushed into the container, the liquid in the container will be squeezed by the membrane tube and flows through the opening at the lower portion of the membrane tube and the membrane, and enters into the membrane tube. Compared with the traditional method, the present invention only needs simple devices such as membrane tubes and containers, and does not require devices such as centrifuges or vacuum pumps In prior art, sample liquid is initially in the membrane tube and then flows thereout; whereas, in the present invention, sample liquid is initially in the container and then flows through the opening of the lower portion of the membrane tube and enters the membrane tube. The two directions that the liquid flows in the membrane tube are opposite. In the present invention, the mechanism for generating the squeezing force is also relatively simple, and is like a syringe, in which a piston rod is pushed into the syringe to form a liquid-tight state with the syringe, and the squeezing force is generated to inject the liquid. In the present invention, the membrane tube is equivalent to the piston rod and the container is equivalent to the syringe, but the front end of the syringe has a hole and the container of the present invention is closed. The opening at the front end of the syringe is changed to the lower portion of the membrane tube. Although the syringe device is different from the present invention, the method of operation is similar. The present invention can replace a traditional centrifuge and vacuum pump by using the simple extrusion device comprising the membrane tube and the container, especially in the simplification of the recovery process, in which the dissolving solution can be left in the original membrane tube and used directly, and no additional container is needed to receive the dissolving solution, so the operation device and processes can be simplified. Moreover, the method of extracting biomolecules from the membrane tube can be more easily implemented on an automated machine.

### BRIEF DESCRIPTION OF THE DRAWINGS

FIG. 1 illustrates a membrane tube;

FIG. 2 illustrates a container;

FIG. 3 illustrates the state of the container filled with liquid; and

FIG. 4 illustrates the state in which the membrane tube is pushed into the container.

### DETAILED DESCRIPTION

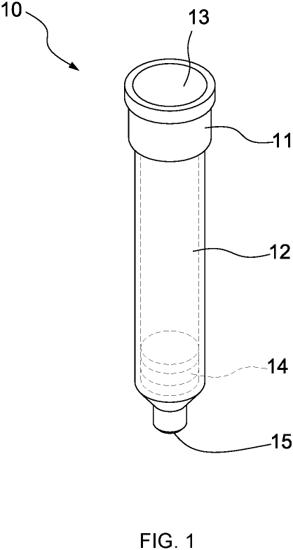

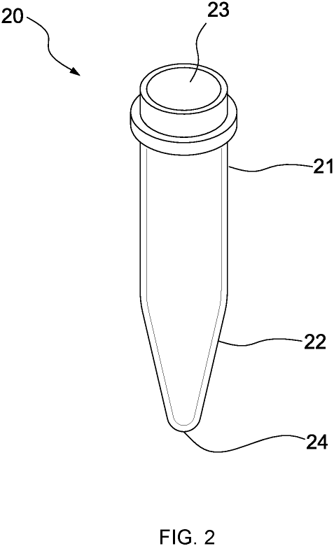

The device for extracting biomolecules of the present invention includes a membrane tube 10 and a container 20. FIGS. 1 and 2 show the membrane tube 10 and the container 20, respectively.

The membrane tube 10 is a common device used in the process of extracting biomolecules, and is roughly the shape of a round tube, and comprises an upper portion 11 and a lower portion 12. The upper portion 11 has an upper opening 13, and the lower portion 12 contains permeable membranes 14, and has a lower opening 15.

FIG. 2 shows a container 20. The container 20 is also roughly tubular, and comprises an upper portion 21 and a lower portion 22. The upper portion 21 of the container has an opening 23, and the lower portion 22 has a closed end 24.

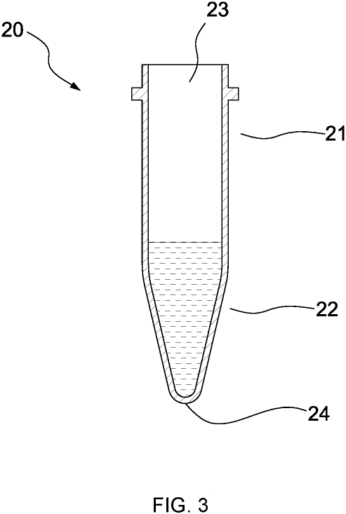

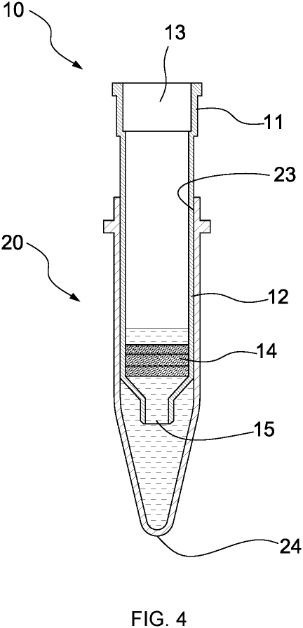

When the device is used, liquid is added to the container 20 (see FIG. 3), and the membrane tube 10 is pushed into the container 20 (see FIG. 4). The liquid in the container 20 is squeezed by the membrane tube 10 to pass through the lower opening 15 of the membrane tube 10 and the permeable membrane 14 and flow into the membrane tube 10.

The membrane tube 10 is configured to adapt to the container 20 so that when it is pushed into the container 20, the interface therebetween must be liquid-tight, and the liquid in the container 20 will not leak through the interface, but will flow through the opening 15 in the lower portion of the membrane tube and through the permeable membrane 14 and enter the membrane tube 10.

The use of the above-mentioned membrane tube 10 and container 20 to extract biomolecules is more suitable for automation than conventional methods. In the recovery procedure of extracting biomolecules, dissolving solution is added into the container 20, and then the membrane tube 10, which contains the permeable membrane 14, is pushed into the container 20 through the upper opening 23 of the container 20. Since it is liquid-tight between the container 20 and the membrane tube 10, the liquid in the container 20 is squeezed by the membrane tube 10 to pass through the lower opening 15 of the membrane tube 10 and the permeable membrane 14, and enter the membrane tube 10.

The present invention is very different from the traditional method in the recovery process. In the traditional method, the dissolving solution is added to the membrane tube; the dissolving solution can detach the biomolecules from the membrane, and then a force (such as centrifugal force or air pressure) is applied to push the dissolving solution out of the membrane tube. A container is provided to receive the dissolving solution that contains the biomolecules to achieve the purpose of recovering target biomolecules. In the method of the present invention, the dissolving liquid is added to the container 20, and then the membrane tube 10 is pushed into the container 20 from the upper opening 23 of the container 20. The solution in the container 20 is thereby squeezed by the membrane tube 10 and flows through the lower opening 15 of the membrane tube 10, the permeable membrane 14, and then into the membrane tube 10. The dissolving liquid (i.e., the recovery liquid) passing through the permeable membrane 14 will be contained in the membrane tube 10; therefore, it is unnecessary to use a centrifuge or a vacuum pump device to collect the dissolving liquid in another new container, and thus the implementation and operation of automation can be simplified. The method of extracting biomolecules from membrane tubes is easier to implement on an automated machine.

The content of the present invention described above is only one of the possible ways to implement the present invention Modifications, replacements, and combinations made for the above-mentioned embodiments can be easily completed by those skilled in the art in the field of the present invention and are within the scope of the inventive concept.

What is claimed is:

1. A device for extracting biomolecules, comprising:
a membrane tube, comprising an upper portion and a lower portion, the upper portion having an upper opening, the lower portion accommodating a permeable membrane, and having a lower opening;
a container, comprising an upper portion and a lower portion, the upper portion having an opening allowing the membrane tube to be pushed into the opening of the upper portion of the container, and a liquid-tight seal is formed between the membrane tube and the container, the lower portion of the container has a closed end.

2. A method for extracting biomolecules using a membrane tube, comprising:
adding an appropriate amount of liquid to the container according to claim 1;
pushing the membrane tube according to claim 1 into the container, whereby the liquid in the container flows through the permeable membrane and enters into the membrane tube.
